(19) United States
(12) Patent Application Publication
(10) Pub. No.: US 2007/0292421 A1
(43) Pub. Date: Dec. 20, 2007

(54) Method for treating preeclampsia

(76) Inventor: Bruce B. Feinberg, Lakewood, NJ (US)

Correspondence Address: Dr. Bruce B. Feinberg, 314 Caranetta Drive, Lakewood, NJ, 08701, US

(21) Appl. No.: 11/477,317

(22) Filed: Jun. 29, 2006

Related U.S. Application Data
(60) Provisional application No. 60/703,186, filed on Jul. 28, 2005.

(51) Int. Cl. A61K 39/395; A61K 39/00; A61P 15/00

(52) U.S. Cl. 424/135.1; 424/133.1

(57) ABSTRACT

Disclosed are methods and compositions for treating or inhibiting the onset of preeclampsia through administration to a pregnant mammal in need of such a treatment an effective amount of at least one C5a inhibitor. The C5a inhibitor may be co-administered with other active agents.

This nonprovisional application claims the benefit of provisional application for patent, U.S. Appln. No.: 60/703,186 filed in the United States Patent and Trademark Office on Jul. 28, 2005.

### STATEMENT REGARDING FEDERALLY SPONSORED RESEARCH OR DEVELOPMENT

Not Applicable.

### REFERENCE TO SEQUENCE LISTING, A TABLE OR COMPUTER PORGRAM LISTING COMPACT DISC APPENDIX

Not Applicable.

### BACKGROUND OF THE INVENTION

Preeclampsia, also known as toxemia, occurs during pregnancy. This condition is characterized in part by high blood pressure, the presence of protein in the urine, swelling (edema) due to fluid retention, abnormal kidney function, excessive weight gain, severe headache, nausea and visual disturbances. Preeclampsia is a leading cause of maternal and neonatal death worldwide. See Goodburn et al., Reducing maternal mortality in the developing world: sector-wide approaches may be the key, Br. Med. J., 322:917-20 (2001). Preeclampsia is also a leading cause of fetal growth restriction, intrauterine fetal demise and indicated preterm birth.

Current treatments for preeclampsia include delivery of the fetus, bed rest, diet management, anti-convulsant medication (to prevent seizures) and blood pressure medication. However, such treatments may have drawbacks. For example, delivery of the fetus is a practical option only at or near term. Additionally, these treatments address certain conditions resulting from or associated with preeclampsia and not the disease itself.

Accordingly, there is a need to develop methods of treating and/or inhibiting the onset of preeclampsia.

### SUMMARY OF THE INVENTION

Thus, in one aspect, the present invention is directed to a method of treating preeclampsia, comprising administering to a pregnant mammal with or at risk of preeclampsia an effective amount of at least one C5a inhibitor. In some embodiments, the C5a inhibitor is co-administered with one or more other active agents.

Another aspect of the present invention is directed to a therapeutic combination or cocktail comprising at least one C5a inhibitor and at least one other active agent, each in an effective amount to treat a pregnant mammal having or at risk of preeclampsia.

### DETAILED DESCRIPTION

Pertinent art and science have speculated inconsistently that preeclampsia is an immune mediated disease. Other literature has led to the general conclusion that inhibition of C5a will likely mitigate the inflammatory response on immune mediated diseases. However, the state of the art definitively does not include the treatment of preeclampsia specifically with C5a inhibition, or a cocktail of drugs to include a C5a receptor blockade among other significant remedial and simultaneous therapies. A relevant reference is Table 1 of U.S. Pat. No. 6,821,956 by David Fairlie (see PTO/SB/08a INFORMATION DISCLOSURE STATEMENT BY APPLICANT) that ubiquitously lists, with no teaching, a plethora of diseases, which have been noted across the span of academic literature to have a possible immune etiology, as potentially treatable by C5a inhibition. The Fairlie patent does not discuss preeclampsia in any detail and does not disclose the cocktail of therapy explicated in the present application for patent which has: 1) a preventative application for those patients at high risk for preeclampsia; and 2) a curative application for those patients in the various acute stages of the disease..

Distinct from what is disclosed in the Fairlie patent, and what distinguishes this invention from the current literature, is the a) finely detailed and unique teaching/understanding of the cause of preeclampsia to be rooted in the slight imbalance of production versus removal of immune complexes and the resultant autoamplification of immune complex production; whereby, even a minimal failure of the maternal immune system to effectively clear trophoblastic debris leads to a net proinflammatory response with a resultant oxidative stress such that the pathophysiologic sequelae, while mild, begin immediately as the balance is tipped in favor of production of immune complexes over removal; and based on this understanding, b) the cocktail of drugs, including but not limited to C5a receptor blockade, designed by the inventor to 1) prevent the disease by inhibiting the autoamplification process before it begins in those patients at high risk for preeclampsia and/or 2)ameliorate the concurrent symptoms of the acute disease across many major organ and circulatory systems in the body once the pathophysiologic sequelae have begun.

See the inventor's article Feinberg, B. B., The Death of Goliath, Am J Reprod Immunol 55:84-98,(2006). The concept that immune complexes are involved in the pathogenesis of preeclampsia is not new. However, despite initial enthusiasm, early observations that immune complex levels in normal pregnancy are often similar to those found in “mild preeclampsia” cast doubt on the preeclampsia/immune complex theory. The inventor distinguishes himself from the community of scientists in purporting that precisely within the statistically similar immune complex levels between normal gestations and mild preeclamptics lies the secret to understanding the pathophysiology of the disease; that is, the onset of the disease is masked behind a deceptive, statistically undetectable change in the production of immune complexes relative the maternal clearance ability.

It is fundamental to understand the generation and processing of immune complexes during human pregnancy. The epidemiology of preeclampsia implicates the placenta as the antigenic source. As part of the normal syncytium turnover placental apoptosis increases significantly as normal pregnancy advances releasing syncytiotrophoblast debris including syncytiotrophoblast microfragments, cell free fetal DNA, and cytoplasmic proteins (e.g., cytokeratin fragments). This cellular debris generates circulating immune complexes capable of initiating a maternal systemic inflammatory response. As long as the maternal capacity to clear these immune complexes keeps pace with production, no pathophysiologic sequelae manifest. However, the nexus between normal pregnancy and disease lies immediately in tipping the balance in favor of production of immune complexes over removal. If the burden of trophoblast debris exceeds the maternal clearance ability, a net “proinflammatory” process ensues with a resultant oxidative stress. This is a critical turning point in the disease process as one of the most reproducible inducers of apoptosis is mild oxidative stress. Hence, this maternal oxidative stress in turn stimulates further placental apoptosis and necrosis generating an autoamplification process of placental apoptosis, trophoblast shedding/deportation, immune complex production, maternal inflammatory response, oxidative stress, further placental apoptosis, etc., ultimately culminating in clinical preeclampsia.

Long before the circulating immune complex levels are statistically different from normal gestations, the resultant preeclamptic inflammatory process is well underway. The shift of the production versus removal balance favoring immune complex excess initially is extremely subtle; nonetheless, the inflammatory consequences have begun. As the disease worsens, the levels of immune complexes become more disparate from normal pregnancies leading to more catastrophic clinical findings.

With the inventor's teaching in mind, one can revisit the prior data on immune complexes in preeclampsia. Taken as a whole, studies evaluating immune complexes in preeclampsia are plagued by lack of uniformity in the clinical definition of preeclampsia and severity of disease, lack of controls, varying assay techniques, age of gestation at sample collection, and logistic issues regarding sample collection and processing procedures. Nonetheless, even with these caveats, on review of these studies an underlying theme emerges: an incremental rise in circulating immune complexes is seen in normal pregnancy, a statistically insignificant rise in the mild preeclamptics vis-a-vis normal gestations, and a significant increase in women with “moderate—severe preeclampsia”.

Complement protein regulation and the inflammatory response: the main immune complex clearance mechanism in the human is via the erythrocyte complement receptor type 1 (CR1, C3b receptor, CD35). Erythrocyte CR1 is a complement regulatory protein which functions primarily as a receptor for C3b-opsonized circulating immune complexes, delivering these complexes to the fixed macrophage system in the liver and spleen for clearance. This sequence of biologic recognition reactions is completed in less than two minutes, emphasizing that the immune adherence phenomenon plays a crucial role in the clearance of immune complexes from the circulation. Erythrocytes express approximately 500 CR1 receptors per cell and account for roughly 90% of all CR1 in the circulation. Although a genetic absence of human CR1 has not been reported, decreases in membrane bound CR1 concentrations, both inherited and acquired, have been observed in various autoimmune diseases such as systemic lupus erythematosis. A low concentration of erythrocyte CR1 limits immune complex handling and thus leads to pathologic immune complex mediated biologic effects. A decreased expression of erythrocyte CR1 in preeclamptic patients correlating with severity of disease has been documented by the inventor: see Feinberg, et al., Low Ertythrocyte Complement Receptor Type 1 (CR1, CD35) Expression in Preeclamptic Gestations, Am J Reprod Immunol 54:352-357,2005).

The inventor's model posits the nexus between normal pregnancy and disease to lie immediately in tipping the balance in favor of production of immune complexes over removal. In normal pregnancies, the autoamplification inflammatory process does not occur since there is sufficient erythrocyte CR1 expression relative to the immune complex load, allowing for removal and processing of the complexes before they exceed the body's handling mechanism and become pathologic. The converse is true in preeclamptic pregnancies.

The strength in this “balance theory” of preeclampsia lies in its ability to comprehensively explain the myriad of clinical expressions of preeclamptic conditions as well as the normal pregnant state (i.e., absence of disease). For example, if low erythrocyte CR1 is matched with low immune complex production, no adverse sequelae would be anticipated. This would explain why some normal pregnant patients with low erythrocyte CR1 expression do not demonstrate preeclamptic sequelae. Similarly, at the opposite extreme are those preeclamptic patients with higher erythrocyte CR1 levels who represent a patient population with excessive immune complex production such as multiple gestations, molar pregnancies, or concurrent disease (e.g., SLE, APLAS). Another compelling observation is in multigravidas, where the “adaptive protection” afforded by a prior pregnancy of the same paternity reflects an immune tolerance phenomenon and a decrease in immune complex production with subsequent pregnancies thus lowering the risk of subsequent preeclampsia. Another persuasive scenario is the oocyte donor pregnancy. Here one would anticipate a greater antigenic load and immune complex production since the entire conceptus is foreign to the surrogate mother, raising the risk for associated preeclampsia.

Diagnostic and Therapeutic Strategies

See Feinberg, B. B., The Death of Goliath, Am J Reprod Immunol 55:84-98,(2006). The diagnostic and therapeutic implications of the inventor's immune complex balance model suggest the inhibition of complement activation as a primary focus and most promising arena for the treatment of preeclampsia to include: C5 binding antibodies; anti C5a blocking antibodies; C5a receptor antagonists. In collusion with neutralizing the effect of C5a, therapeutic treatment should include: mechanisms to decrease apoptosis; modulation of the immune/inflammatory response; inhibition of granulocyte activation; inhibition of coagulation; antioxidant therapy; serotonin/histamine blockade; inhibition of platelet activation. The inventor also distinguishes himself in his recommendations of therapy for the acute treatment of severe preeclampsia/HELLP syndrome remote from term as well as preventive strategies for patients at risk for the development of preeclampsia.

The pivotal variable involved in the systemic manifestations of the preeclampsia is the generation of complement anaphylatoxins by activation of the classical complement cascade. Numerous animal models and limited human data have demonstrated the efficacy of complement regulators in the treatment of inflammatory disease. Pharmacologic strategies for regulation of complement have maneuvered around two main points: a) site in the complement cascade to regulate, and b) the agent developed (i.e., monoclonal antibodies vs. small molecule inhibitors which offer manufacturing and delivery advantages). For example, while C5a is at least ten times more potent than C3a in inducing biological responses, the concentration of C3/C3a is 15 times higher in plasma than C5/C5a, leading some to suggest that inhibition of complement at the C3 level may be more effective. The most promising monoclonal antibody inhibitor of C3 is soluble CR1 (TP-10, Avant Immunotherapeutics, Needham, Mass.). A potent small molecule alternative is a cyclic peptide C3 inhibitor, Compstatin, (John D. Lambris PhD, University of Pennsylvania). However, it may be impractical and potentially dangerous to induce a complete complement deficient state, whereas blockade of C5, C5a, or C5a receptor may be a more suitable therapeutic approach. Blocking the complement cascade at C5 inhibits mediators and effectors of tissue injury while preserving the complement derived immunoprotective effects of C3. Antibodies which block the generation of C5a, specific anti-C5a blocking antibodies, and C5a receptor antagonists are all capable of attenuating multi-organ injury in experimental models.

a) C5 Binding Antibodies

Pexelizumab and eculizumab (Alexion Pharmaceuticals, Cheshire, Conn.) are recombinant antibody fragments that target and bind to human C5, blocking the cleavage of C5 by C5 convertase enzymes, and thus, blocking the generation of C5a. Clinical studies to date demonstrate the safety and efficacy of these novel anti-inflammatory molecules. Pexelizumab is short acting with elimination half life of 7.0-14.5 hours, the latter after a 2 mg/kg dose, whereas eculizumab is a long acting molecule. One might speculate a role for pexelizumab in the acute treatment of severe preeclampsia remote from term, whereas eculizumab might offer a protective/preventive role in averting the inflammatory symptoms of preeclampsia in patients identified early in pregnancy at risk for the disease.^{Error Bookmark not defined}.

b) Anti C5a Blocking Antibodies

Another approach to neutralizing the effects of C5a is specific blocking antibodies to the C5a molecule. Here the C5a moiety of C5 is bound and neutralized without interfering with C5 cleavage and the subsequent formation of the lytic C5b-9 membrane attack complex. One of the more promising inhibitors in this group is Mab 137-26 (Michael Fung, Tanox Inc, Houston, Tex.).

c) C5a Receptor Antagonists

The University of Queensland, Australia (David Fairlie and Stephen Taylor, Institute for Molecular Bioscience) has generated a potent, orally active inhibitor of the human C5a receptor, known as 3D53 (Ac-Phe [Orn-Pro-D-Cha-Trp-Arg]). This compound is a macrocycle peptidomimetic of the human plasma protein C5a and displays excellent anti-inflammatory activity in numerous models of human disease. In phase II clinical trials, 3D53 demonstrated superior efficacy to NSAID's and glucocorticoids in decreasing inflammatory sequelae and displayed little or no toxicity. That the molecule is orally active makes it potentially an ideal agent for long term preventive therapy of inflammatory disease from preeclampsia. Another C5a receptor blocker, NGD 2000-1 (Neurogen Corporation, Branford, Conn.), unfortunately was tabled after failure to demonstrate clinical efficacy in primary endpoints in both asthma and rheumatoid arthritis patients. This molecule however, may have implications in treatment for preeclampsia.

Mechanisms to Decrease Apoptosis

Apoptosis is a highly regulated process of programmed cell death which is essential to the health and homeostasis of a given tissue by eliminating superfluous, damaged, mutated, or aged cells. The process is orchestrated by the activation of cysteine aspartate-specific proteases, or caspases, via two distinct signaling pathways: a mitochondrial-cytochrome C (receptor independent) pathway, and a ligand-death receptor dependent pathway. In the ligand-death receptor pathway, ligation of death receptors induces the formation of a death inducing signaling complex (DISC) resulting in apoptosis. Innate regulation of apoptosis is via separate pro- and anti-apoptotic members of the Bcl-2 protein family in the mitochondrial pathway. Also, natural inhibitors of caspases, coined the “inhibitors of apoptosis proteins (IAPs)”, are found in cells and exhibit anti-apoptotic activity to a broad range of stimuli in both pathways.

In pregnancies complicated by preeclampsia an increased level of placental apoptosis is demonstrated. Thus, key effector molecules in this process, such as caspases, select anti-apoptotic Bcl-2 proteins, IAPs (e.g., Smac/DIABLO and survivin), and DISC components are promising targets for pharmacologic modulation of apoptosis. Many new therapeutic agents in apoptosis regulation are already well under development as anti-cancer approaches. (Table xx). Application of these agents to patients presenting with severe preeclampsia may be a novel and effective use of these drugs. By decreasing placental apoptosis in cases of advanced clinical preeclampsia, the immune complex production side of the balance would be decreased resulting in a lower proinflammatory burden for the mother.

Another approach in attenuating placental apoptosis is the use of heparin and aspirin. Clinically, heparin and aspirin are widely used with success as treatment for pregnant patients with APLAS. In in vitro studies, heparin and aspirin regulate trophoblast apoptosis. The clinical benefits of these agents may thus exceed their known effects on inhibition of coagulation and thrombosis by decreasing the maternal trophoblast apoptotic burden. The concept of regulated apoptosis in the treatment of preeclampsia warrants further investigation.

Mechanisms to Decrease Apoptosis

The natural means of achieving a decrease in immune complex production are either delivery of the placenta (which currently remains the only known cure to preeclampsia), or having more children with the same paternity (i.e., induction of immune tolerance in multigravidas. Medical therapy to decrease the immune complex load, in theory, might include agents to decrease maternal antibody production. Pharmacologic interruption of B cell antibody production can be accomplished via CD20 monoclonal antibodies. For example, Rituximab (Roche Pharmaceuticals, Nutley, N.J.) is a monoclonal antibody in clinical use for treatment of autoimmune disease and several types of non-Hodgkin's lymphoma. The antibody binds to the B cell surface protein CD20 triggering the body's immune system to attack and destroy the cell. Since normal B cells are quickly replaced toxicity is low. In the case of preeclampsia, one might conjecture that low dose Rituximab may decrease the B cell antibody load enough to achieve a decrease in immune complex production. In two small series (n=11 patients combined) of antineutrophil cytoplasmic antibody (ANCA) positive patients, the addition of Rituximab to the immunosuppressive drug regimen resulted in a clinical improvement in all eleven cases, eight of which were complete.

Modulation of the Immune/Inflammatory Response

Pharmacologic means of attenuating the maternal inflammatory response include corticosteroid administration. Steroids act at the genetic level and result in down-regulation of immune pathways and various proinflammatory mediators, such as cytokines. In clinical trials, several small studies evaluating the effects of corticosteroids on maternal and neonatal mortality and morbidity in women with HELLP syndrome were summarized in a Cochrane review. “Of the five studies reviewed (n=170), three were conducted antepartum and two postpartum. Of the secondary maternal outcomes, there was a tendency to a greater platelet count increase over 48 hours, statistically significantly less mean number of hospital stay days, and mean interval (hours) to delivery in favor of women allocated to dexamethasone. In addition, women randomized to dexamethasone fared significantly better for: oliguria, mean arterial pressure, mean increase in platelet count, mean increase in urinary output and liver enzyme elevations. The mean birthweight was significantly greater in the group allocated to dexamethasone. (While) there were no significant differences in the primary outcomes of maternal mortality and morbidity due to placental abruption, pulmonary edema and liver hematoma or rupture, or in perinatal mortality or morbidity due to respiratory distress syndrome, need for ventilatory support, intracerebral hemorrhage, necrotizing enterocolitis and a five minute Apgar less than seven,” these data regarding secondary outcome measures lend credence to the immune modulating effects of corticosteroids in severe maternal preeclamptic disease and should not be dismissed. These drugs, however, act non-specifically, have undesirable side effects, and their chronic use must be considered only with reserve.

Additional targets for the modulation of immune responses in preeclampsia might include anti-inflammatory agents and monoclonal anti-cytokine antibodies. For example, TNF-a antibodies, soluble TNF receptors (causing TNF inhibition), and interleukin-1 receptor antagonists are under clinical trials in the treatment of sepsis associated systemic inflammatory response. Exploring these treatments for preeclampsia is uncharted. Some reports have noted a deficiency of placental and serum inhibitory cytokine levels, such as interleukin-10, in preeclampsia. Thus, the addition of inhibitory cytokines such as interleukin-lo, may be of value in treatment. However, at high doses IL-10 paradoxically has proinflammatory effects potentially limiting its clinical utility.

Inhibition of Granulocyte Activation

Traditionally immune complexes were thought to procure inflammatory effects only via complement activation. More recently, it has become clear that direct activation of effector cells by immune complexes is intricately involved in their inflammatory sequelae, with granulocyte Fcg receptors playing the pivotal role in this pathway. Fcg receptors are surface glycoproteins, members of the immunoglobulin gene superfamily of proteins, that can bind the Fc portion of immunoglobulin molecules. Fcg receptor expression is under the redundant control of numerous cytokines and genetic factors. Both activating and inhibitory signals are transduced through the Fcg receptors following ligation. These diametrically opposing functions result from structural differences among the different receptor isoforms. Two distinct domains within the cytoplasmic signaling domains of the receptor called immunoreceptor tyrosine based activation motifs (ITAMs) or immunoreceptor tyrosine based inhibitory motifs (ITIMs) account for the different responses. The recruitment of different cytoplasmic enzymes to these structures dictates the outcome of the Fcg receptor-mediated cellular responses. The balance between activation proinflammatory receptors (FcgRI and FcgRIII) and inhibition receptors (FcgRIIB) is critical to the net immune response. Upregulation of the activation FcgRIII, induced by IFN-g or C5a; results in lowered threshold for immune complex stimulation and consequently an enhanced inflammatory response. Conversely, upregulation of the inhibitory FcgRIIB molecule raises the threshold for immune complex stimulation and suppresses inflammatory response to IgG antibodies. More recent data implicate the cell surface density of FcgRIIB as potentially the immune response “gatekeeper” in this balance. From this model one would predict that upregulating inhibitory FcgRIIB should result in protection from immune complex mediated injury. Indeed, pharmacologic administration of intravenous immune globulin (IVIG) induces inhibitory FcgRIIB expression, thus raising the threshold for immune complexes to trigger FcgRIII activation. To underscore this point, Branch et. al. performed a pilot study to determine the impact of intravenous immune globulin on obstetric and neonatal outcomes among women with antiphospholipid syndrome. The findings of fewer cases of fetal growth restriction and neonatal intensive care unit admissions among the intravenous immune globulin-treated pregnancies suggested expansion of the study

In addition, the complement system itself can influence Fcg receptor activity. Both complement and IgG Fc receptors interact in vivo with C5a acting as a early regulator of the induction of activating FcgRIII and suppression of the inhibitory FcgRII. Thus, regulation of activation FcgRIII by C5a blockade, or conversely, pharmacologic upregulation of inhibition FcgRIIB by IVIG may serve to alter the threshold of immune complex mediated inflammation and injury. Other strategies to influence Fcg receptor activity might include statin therapy and Fcg receptor specific antibodies (MacroGenics Inc, Rockville, Md.).

Inhibition of Coagulation

At rest the endothelial surface is essentially non-thrombogenic. This state is largely maintained by tissue factor pathway inhibitor (TFPI) which blocks the initiation of blood coagulation by tissue factor. Endothelial cells are the main source of TFPI. There exists an intricate interrelationship between the coagulation system and host inflammatory response. For example, inflammatory cytokines can activate coagulation and inhibit fibrinolysis, whereas thrombin is able to stimulate multiple inflammatory pathways. The coagulation cascade is activated in patients with preeclampsia. With severe maternal preeclamptic disease a marked consumptive coagulopathy and thrombopathy can manifest as disseminated intravascular coagulation. Potential strategies to control the progression of procoagulant activity include the mainstays of heparin and low dose aspirin. Newer antithrombotic agents, including recombinant TFPI (tifacogin, Chiron Corp/Pharmacia Corp) and recombinant activated protein C (drotrecogin alfa/Xigris, Eli Lilly, Indianapolis) are under clinical evaluation in patients with severe sepsis. The OPTIMIST trial showed a failure of tifacogin in the treatment of severe sepsis. The results of Xigris in the PROWESS trial demonstrated significant reduction in mortality, though an increased risk of severe bleeding. Until further data suggest a reason to switch to these newer agents, treatment with heparin will likely remain the logical and less expensive consideration. A promising new oral anticoagulant is ximelagatran/Exanta (AstraZeneca, Waltham, Mass.). Exanta is a direct thrombin inhibitor and is as effective as enoxaparin/warfarin in the treatment of deep venous thrombosis. Ximelagatran does not require routine coagulation monitoring or dose adjustment, though a spurious elevation of alanine aminotranserase occurs in approximately 10% of patients. There are no data available to date for the use of this drug in pregnancy.

Antioxidant Therapy

Preeclampsia is associated with an increased production of reactive oxygen species as a result of the inherent ongoing inflammatory process. Adjuvant administration of antioxidant therapy would be postulated to aid in amelioration of clinical symptoms. In one randomized trial supplementation with daily Vitamin C (1000 mg) and Vitamin E (400 IU) was associated with a 54% reduction in the rate of preeclampsia in women identified as being at high risk for preeclampsia by abnormal uterine artery Doppler analysis or prior disease history. This antioxidant therapy was also associated with improvement in the biochemical indices of preeclampsia. While this study demonstrated a 54% reduction in preeclampsia, a reproducible reduction in incidence of preeclampsia to this degree is likely generous and perhaps a more realistic projection would be in the 25%-33% range. Nonetheless, as part of a therapeutic strategy, adjuvant therapy with antioxidants is essential to the therapeutic “cocktail” for preeclampsia.

Serotonin/Histamine Blockade

Basophils and mast cells serve as central mediators in the inflammatory response. They are stimulated to degranulate by C5a and immune complexes and release an array of inflammatory mediators including histamine and cytokines. H1 receptor blockers (e.g., loratadine) exhibit anti-inflammatory effects that extend beyond histamine blockade at the H1 receptor such as inhibition of cytokine generation. Serotonin, another potent mediator of inflammation, is released by platelet activation. Serotonin is associated with vasoconstriction of various vascular beds including the uterine and placental circulations. Another effect is endothelial cell retraction which predisposes to increased vascular permeability and clinical edema. Cyproheptadine (PeriActin), a combined serotonin—histamine blocker, may prove useful in combination therapy for preeclampsia.

Inhibition of Platelet Activation

The most commonly investigated antiplatelet agent evaluated to date is low dose aspirin. In a recent Cochrane review the risk of preeclampsia associated with the use of antiplatelet drugs decreased by 15% and the risk of neonatal mortality decreased 14%. The authors concluded that antiplatelet agents, principally low dose aspirin, have small to moderate benefits in the prevention of preeclampsia. The difficulty to date in instituting low dose aspirin preventive therapy has stemmed from the lack of a reliable predictive test for the development of preeclampsia. However, with the development of proper screening measures for the subsequent development of preeclampsia, low dose aspirin will be a useful agent in the combination therapy approach to prevention of the disease.

Therapeutic Strategies

Acute treatment of severe preeclampsia/HELLP syndrome remote from term.

Clearly at term the proper management of preeclampsia should remain delivery. However, in cases of marked prematurity, temporizing measures to ameliorate to inflammatory burden may be considered. From the points made above, one might consider combination therapy with either soluble CR1 (TP-10, Avant Immunotherapeutics) or a C5 blockade (Pexelizumab, Alexion Pharmaceuticals) for complement regulation, intravenous immune globulin for upregulation of the inhibitory FcgRIIB, corticosteroids for immune modulation, an anti-hypertensive agent (e.g., Ketanserin, Labetalol, or nifedipine, low molecular weight heparin/low dose aspirin for inhibition of the clotting cascade as well as inhibition of placental apoptosis, antioxidant (vitamin C & E) therapy, and histamine/serotonin blockade. The role of anticytokines needs to be investigated further. Preventive strategies for patients at risk for the development of preeclampsia.

A number of screening tests have been proposed as markers of increased risk of preeclampsia without sufficient reliability. More recently a number of newer markers have been proposed including: log[sFlt-1/P1GF] ratio, placental growth factor, cell free fetal DNA concentration, uterine artery Doppler velocimetry, PAPP-A levels, erythrocyte CR1 levels, and breath markers of oxidative stress. Once a proper screen is demonstrated, then given the balance model of immune complex production versus removal, one might consider instituting a regimen including: heparin/low dose aspirin vs. Exanta, 3D53—an orally active C5a receptor blocker vs. weekly eculizumab injection, antioxidants (vitamins C & E), serotonin/histamine blockade, and possible administration of steroids (e.g., prednisone).

As used herein, a C5a inhibitor is any agent (e.g., compound, molecule or polymer) that blocks or inhibits activation of complement C5a in the complement cascade. This blocking may be in the form of binding to the complement C5 to prevent cleavage of complement C5 and generation of complement proteins C5a and C5b. The inhibitors may inactivate (e.g., bind directly to) free complement C5a itself. In other embodiments, the inhibitor binds the C5a receptor, and thus antagonizes or interferes with the binding of complement C5a to the C5a receptor. Therefore, C5a inhibitors useful in the present invention include but are not limited to agents such as antibodies that specifically bind the C5a moiety of complement C5 and/or free complement C5a, and agents that specifically bind the C5a receptor.

As used herein, the term “antibodies” refers to immunoglobulins produced in vivo, as well as those produced in vitro by a hybridoma, antigen binding fragments (e.g., Fab′ preparations) of such immunoglobulins, as well as to recombinantly expressed antigen binding proteins, including immunoglobulins, chimeric immunoglobulins, “humanized” immunoglobulins, antigen binding fragments of such immunoglobulins, single chain (e.g., of variable light and heavy fragments) of antibodies, and other recombinant proteins containing antigen binding domains derived from immunoglobulins. Publications describing methods for the preparation of such antibodies include Reichmannet al., Nature 332:323-327 (1988); Winter and Milstein, Nature 349:293-299 (1991); Clacksonet al., Nature 352:624-628 (1991); Morrison, Annu Rev Immunol 10:239-265 (1992); Haber, Immunol Rev 130:189-212 (1992); and Rodrigueset et al., J Immunol 151:6954-6961 (1993). The antibodies used in the present invention are preferably monoclonal antibodies (MAbs). Monoclonal antibodies may be made using the hybridoma method first described by Kohler et al., Nature 256:495 (1975), or by other methods known in the art.

In the present invention, the antibodies are preferably “humanized.” A “humanized” antibody is designed to have greater homology to a human immunoglobulin than animal-derived antibodies. Non-human amino acid residues from an “import” (animal) variable domain are transfected into a human “backbone.” Humanization can be essentially performed following the methods reported in Jones et al., Nature 321:522-525 (1986); Riechmann et al., Nature 332:323-327 (1988); and Verhoeyen et al., Science 239:1534-1536 (1988), by substituting rodent complementarity determining regions (“CDRs”) or CDR sequences for the corresponding sequences of a human antibody. Accordingly, in such “humanized” antibodies, the CDR portions of the human variable domain are substituted by the corresponding sequence from a non-human species. Thus, humanized antibodies are typically human antibodies in which some CDR residues and possibly some framework residues are substituted by residues from analogous sites in rodent antibodies.

The choice of human variable domains, both light and heavy domains, to be used in making the humanized antibodies is important in order to reduce antigenicity. According to the so-called “best-fit” method, the sequence of the variable domain of a rodent antibody is screened against the entire library of known human variable-domain sequences. The human sequence that is closest to that of the rodent is then accepted as the human framework (FR) for the humanized antibody (Sims et al., J. Immunol. 151:2296 (1993); Chothia et al., J. Mol. Biol. 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA 89:4285 (1992); Presta et al., J. Immunol. 151:2623 (1993)).

To ensure that humanized antibodies retain high affinity for the antigen, they may be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of certain residues in the functioning of the candidate immunoglobulin sequence, i.e., the analysis of residues that influence the ability of the candidate immunoglobulin to bind its antigen. In this manner, FR residues can be selected and combined from the recipient and import sequences so that the desired antibody characteristic, such as increased affinity for the target antigen(s), is maximized, although it is the CDR residues that directly and most substantially influence antigen binding.

One can also produce transgenic animals (e.g., mice) that are capable, upon immunization, of producing a full repertoire of human antibodies in the absence of endogenous immunoglobulin production. Such transgenic mice are available from Abgenix, Inc., Fremont, Calif., and Medarex, Inc., Annandale, N.J. It has been described that the homozygous deletion of the antibody heavy-chain joining region (IH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA 90:2551 (1993); Jakobovits et al., Nature 362:255-258 (1993); Bruggermann et al., Year in Immunol. 7:33 (1993); and Duchosal et al., Nature 355:258 (1992). Human antibodies can also be derived from phage-display libraries (Hoogenboom et al., J. Mol. Biol. 227: 381 (1991); Marks et al., J. Mol. Biol. 222:581-597 (1991); Vaughan et al., Nature Biotech 14:309 (1996)).

As indicated above, the antibodies of the present invention may be “native” antibodies or antibody fragments. Native antibodies are full-length immunoglobulin sequences that have not been truncated (e.g., to produce Fv or Fab) or mutated (e.g., spliced to form a chimeric or humanized antibody). The C5a inhibitors may also be “single chain” antibodies. Recombinant DNA methods may be used to construct antibodies that have their heavy (H) and light (L) chains joined by a linker peptide to form a single chain (sc) antibody. As described below, there are several types of sc antibodies that can be constructed.

As is the case for humanization, the effects on antigen binding properties of constructing a particular type of sc antibody using H and L chains that have not been characterized with regard to their ability to function as part of an sc antibody cannot be reliably predicted by any known method. However, the successful construction of any one type of sc antibody from a particular native antibody provides information that, in general, facilitates the successful construction of other types of sc antibodies from the native antibody.

Typically, native antibodies contain one type of L chain and one type of H chain, which are held together by disulfide bonds to form a heterodimeric subunit. The first domain of each chain is highly variable in amino acid sequence, providing the vast spectrum of antibody binding specificities found in each individual. These are known as variable heavy (VH) and variable light (VL) domains. The second and subsequent (if any) domains of each chain are relatively invariant in amino acid sequence. These are known as constant heavy (CH) and constant light (CL) domains.

However, single chain antibodies may include one each of only (variable heavy) VH and (variable light) VL domains, in which case they are referred to as scFv antibodies; they may include only one each of VH, VL, CH, and CL domains, in which case they are referred to as scFab antibodies; or they may contain all of the variable and constant regions of a native antibody, in which case they are referred to as full length sc antibodies. scFv and scFab antibodies with more than one chain are referred to as Fv and Fab antibodies respectively.

The differing sizes of these antibodies impart each with differing pharmacokinetic properties. In general, smaller proteins are cleared from the circulation more rapidly than larger proteins of the same general composition. Thus, full-length sc antibodies and native antibodies generally have the longest duration of action, scFab antibodies have shorter durations of action, and scFv antibodies have even shorter durations of action. Of course, depending upon the illness being treated, longer or shorter acting therapeutic agents may be desired. For example, therapeutic agents for use in the prevention of immune and hemostatic disorders associated with extracorporeal circulation procedures (which are typically of brief duration) are preferably relatively short acting, while antibodies for the treatment of long-term chronic conditions (such as inflammatory joint disease) are preferably relatively long acting. In the case of treating preeclampsia, long acting antibodies are preferred for prophylactic treatment and short acting antibodies are preferred for therapeutic treatment.

Detailed discussions of antibody engineering may be found in numerous publications, including: Borrebaek, Antibody Engineering, A Practical Guide, W.H. Freeman and Co., NY (1992); and Borrebaek, “Antibody Engineering,” 2nd ed., Oxford University Press, NY, Oxford (1995).

One suitable class of C5a inhibitors are those that specifically bind the C5a moiety of native complement C5 (hereinafter “C5”). This binding prevents or at least inhibits cleavage of C5 by C5 convertase enzymes, and inhibits or blocks generation of complement C5a (hereinafter “C5a”) and complement C5b (hereinafter “C5b”).

Therefore, C5 binding antibodies suitable for use in the present invention include the monoclonal antibody fragments described in U.S. Pat. Nos. 6,074,642 and 6,355,245. Preferred C5 binding antibodies of this type are 5G1.1 or h5G1.1 (Eculizumab) and h5G1.1-SC or h5G1.1-scfv (Pexelizumab) (Alexion Pharmaceuticals), which are multi-chain and single-chain fragments of recombinant monoclonal antibodies.

As reported in U.S. Pat. No. 6,355,245, the 5G1.1 antibody is produced from the hybridoma having ATCC Deposit designation HB-1162S. The 5G1.1 hybridoma was obtained according to teachings in U.S. Pat. No. 5,135,916. U.S. Pat. No. 6,355,245 also discloses derivatives of 5G1.1, such as single-chain (i.e., sc) forms and single chain fragments (e.g., scFv and scFab) of 5G1.1. All of the SG1.1-based monoclonal antibodies disclosed in U.S. Pat. No. 6,355,245, whether in single-chain, humanized, full-length or shortened form, share the following characteristics with native 5G1.1, namely: (1) they compete with 5G1.1 for binding to specific portions of C5 that are specifically immunoreactive with 5G1.1; (2) they specifically bind to the foregoing specific portions of C5 (such specific binding, and competition for binding can be determined by various methods well known in the art, including the plasmon surface resonance method (Johne et al., J. Immunol. Meth. 160:191-198(1993)) and (3) they block the binding of C5 to either C3 or C4 (which are components of C5 convertase). These 5G1.1-based monoclonal antibodies, however, do not bind free C5a.

As disclosed in U.S. Pat. No. 6,355,245, the amino acid sequence for a humanized 5G1.1 scFv is shown below in SEQ ID NO:1. Other amino acid sequences of variations of 5G1.1 are also disclosed in U.S. Pat. No. 6,355,245.

SEQ ID NO:1

| | |
|---|---|
| | ATG GCC GAT ATC CAG ATG ACC CAG TCC CCG |
| 30 | |
| | Met Ala Asp Ile Gln Met Thr Gln Ser Pro |
| | 1 5 10 |
| | TCC TCC CTG TCC GCC TCT GTG GGC GAT AGG |
| 60 | |
| | Ser Ser Leu Ser Ala Ser Val Gly Asp Arg |
| | 15 20 |
| | GTC ACC ATC ACC TGC GGC GCC AGC GAA AAC |
| 90 | |
| | Val Thr Ile Thr Cys Gly Ala Ser Glu Asn |
| | 25 30 |
| | ATC TAT GGC GCG CTG AAC TGG TAT CAA CGT |
| 120 | |
| | Ile Tyr Gly Ala Leu Asn Trp Tyr Gln Arg |
| | 35 40 |
| | AAA CCT GGG AAA GCT CCG AAG CTT CTG ATT |
| 150 | |
| | Lys Pro Gly Lys Ala Pro Lys Leu Leu Ile |
| | 45 50 |
| | TAC GGT GCG ACG AAC CTG GCA GAT GGA GTC |
| 180 | |
| | Tyr Gly Ala Thr Asn Leu Ala Asp Gly Val |
| | 55 60 |
| | CCT TCT CGC TTC TCT GGA TCC GGC TCC GGA |
| 210 | |
| | Pro Ser Arg Phe Ser Gly Ser Gly Ser Gly |
| | 65 70 |
| | ACG GAT TTC ACT CTG ACC ATC AGC AGT CTG |
| 240 | |
| | Thr Asp Phe Thr Leu Thr Ile Ser Ser Leu |
| | 75 80 |
| | CAG CCT GAA GAC TTC GCT ACG TAT TAC TGT |
| 270 | |
| | Gln Pro Glu Asp Phe Ala Thr Tyr Tyr Cys |
| | 85 90 |
| | CAG AAC GTT TTA AAT ACT CCG TTG ACT TTC |
| 300 | |
| | Gln Asn Val Leu Asn Thr Pro Leu Thr Phe |
| | 95 100 |
| | GGA CAG GGT ACC AAG GTG GAA ATA AAA CGT |
| 330 | |
| | Gly Gln Gly Thr Lys Val Glu Ile Lys Arg |
| | 105 110 |
| | ACT GGC GGT GGT GGT TCT GGT GGC GGT GGA |
| 360 | |
| | Thr Gly Gly Gly Gly Ser Gly Gly Gly Gly |
| | 115 120 |
| | TCT GGT GGT GGC GGT TCT CAA GTC CAA CTG |
| 390 | |
| | Ser Gly Gly Gly Gly Ser Gln Val Gln Leu |
| | 125 130 |
| | GTG CAA TCC GGC GCC GAG GTC AAG AAG CCA |
| 420 | |
| | Val Gln Ser Gly Ala Glu Val Lys Lys Pro |
| | 135 140 |
| | GGG GCC TCA GTC AAA GTG TCC TGT AAA GCT |
| 450 | |
| | Gly Ala Ser Val Lys Val Ser Cys Lys Ala |
| | 145 150 |
| | AGC GGC TAT ATT TTT TCT AAT TAT TGG ATT |
| 480 | |
| | Ser Gly Tyr Ile Phe Ser Asn Tyr Trp Ile |
| | 155 160 |
| | CAA TGG GTG CGT CAG GCC CCC GGG CAG GGC |
| 510 | |
| | Gln Trp Val Arg Gln Ala Pro Gly Gln Gly |
| | 165 170 |
| | CTG GAA TGG ATG GGT GAG ATC TTA CCG GGC |
| 540 | |
| | Leu Glu Trp Met Gly Glu Ile Leu Pro Gly |
| | 175 180 |
| | TCT GGT AGC ACC GAA TAT ACC GAA AAT TTT |
| 570 | |
| | Ser Gly Ser Thr Glu Tyr Thr Glu Asn Phe |
| | 185 190 |
| | AAA GAC CGT GTT ACT ATG ACG CGT GAC ACT |
| 600 | |
| | Lys Asp Arg Val Thr Met Thr Arg Asp Thr |
| | 195 200 |
| | TCG ACT AGT ACA GTA TAC ATG GAG CTC TCC |
| 630 | |
| | Ser Thr Ser Thr Val Tyr Met Glu Leu Ser |
| | 205 210 |
| | AGC CTG CGA TCG GAG GAC ACG GCC GTC TAT |
| 660 | |
| | Ser Leu Arg Ser Glu Asp Thr Ala Val Tyr |
| | 215 220 |
| | TAT TGC GCG CGT TAT TTT TTT GGT TCT AGC |
| 690 | |
| | Tyr Cys Ala Arg Tyr Phe Phe Gly Ser Ser |
| | 225 230 |
| | CCG AAT TGG TAT TTT GAT GTT TGG GGT CAA |
| 720 | |
| | Pro Asn Trp Tyr Phe Asp Val Trp Gly Gln |
| | 235 240 |
| | GGA ACC CTG GTC ACT GTC TCG AGC TGA |
| 747 | |
| | Gly Thr Leu Val Thr Val Ser Ser |
| | 245 |

Typically, doses of these C5 antibodies, e.g., the 5G1.1 multi and single chain antibodies and derivatives thereof, range from about 1 mg/kg to about 100 mg/kg, and preferably from about 5 mg/kg to about 50 mg/kg with a target plasma concentration of about 35 μg/ml.

Another class of suitable C5a inhibitors includes antibodies that bind with high specificity (specifically binds) to the C5a moiety of C5 and free C5a, and do not prevent cleavage of C5 into C5a and C5b.

One such C5a inhibitor is MAb 137-26, which is disclosed in U.S. Patent Application Publication 2003/0129187 (Fung et al.). MAb 137-26 is produced from the hybridoma deposited with the Accession No. PTA-3650. MAb 137-26 binds to a shared epitope on human C5 and C5a. MAb is capable of binding to C5 before it is activated. Through such a binding, MAb 137-26 does not inhibit the cleavage of C5 to form C5a and C5b, but remains bound to C5a after the cleavage to inhibit the binding of C5a to C5aR, thereby neutralizing C5a. See also Fung et al., Clin. Exp. Immunol., 133:160-169 (2003), which teaches that MAb 137-26 inhibits C5a activation by binding the C5a moiety before cleavage of C5 into C5a and C5b, but does not effect the formation of C3, which is upstream from the C5 step in the complement cascade, or the formation of complement C5b-9, which is responsible for the lysis or killing of bacteria. In this manner, MAb 137-26 binds and neutralizes the C5a moiety without substantially interfering with C5 cleavage or the subsequent formation of the lytic C5b-9 membrane attack complex. According to Fung, MAb 137-26 is also capable of binding to free C5a, or C5a.

Typically, doses for these C5a inhibitors, e.g., MAb 137-26, range from about 0.01 mg/kg to about 50 mg/kg, and preferably from about 0.1 mg/kg to about 10 mg/kg.

Based on the molecular structures of the variable regions of the antibodies, molecular modeling and rational molecular design may be used to generate and screen small molecules that mimic the molecular structures of the binding region of the antibodies and inhibit C5 or free C5a. These small molecules can be peptides, peptidomimetics, oligonucleotides, or organic compounds. Alternatively, large-scale screening procedures are commonly used in the field to isolate suitable small molecules form libraries of combinatorial compounds.

Another suitable class of C5a inhibitors of the present invention is C5a receptor antagonists. The term “C5a receptor” is understood in the art to mean the sites on the surfaces of blood cells, such as PMNLs (polymorphonuclear leukocytes) and monocytic cells, to which C5a and its degradation product C5a-desArg bind. See, for example, U.S. Pat. No. 5,177,190 and Oppermann et al, J. Immunol. 151(7):3785-3794 (1993). In humans, C5a is converted enzymatically to C5a-desArg in human serum by a carboxypeptidase B-like enzyme, and is a major physiological end product in humans. Chenoweth et al., Mol. Immunol. 17:151-161 (1980). By the term “antagonist,” it is meant that these agents interfere with the binding of C5a to the C5a receptor, and thus reduce downstream agonist activity.

Suitable antagonists include “peptidomimetic” compounds, which are generally compounds with “chemical structures derived from bioactive peptides which imitate natural molecules.” See, e.g., Murray Goodman and Seonggu Ro, “Peptidomimetics for Drug Design” chapter twenty in Burger's Medicinal Chemistry and Drug Discovery, Volume 1: Principles and Practice, Manfred E. Wolff, ed. John Wiley & Sons, Inc., NY, 1995, pp. 801-861). As used herein, the term “peptidomimetic” additionally comprises peptoid compounds, which are compounds that comprise oligomers of N-substituted natural amino acids, and the term further comprises any compound having more than two amide bonds. One suitable C5a receptor antagonist, 3D53 (Ac-Phe [Orn-Pro-D-Cha-Trp-Arg]) is reported in Reid et al., A convergent solution-phase synthesis of the macrocycle Ac-Phe-[Orn-Pro-D-Cha-Trp-Arg], a potent new anti-inflammatory drug. J. Org. Chem. 68:4464-71 (2004). In Reid, 3D53 is described as a macrocycle peptidomimetic form of the human plasma protein C5a that displays excellent anti-inflammatory activity in numerous models of human disease. The molecular formula for 3D53 is given in U.S. Pat. No. 6,821,950. The compound 3D53 is orally active with little toxicity, making it a preferred C5a inhibitor for long-term preventive therapy of inflammatory disease from preeclampsia.

Peptidomimetic forms of other C5a inhibitors disclosed herein, in particular MAb 137-26, may also be useful. These peptidomimetic compounds may be selected and made by methods known in the art. The peptidomimetics may be synthesized on a solid support by known techniques (see, e.g., Stewart et al., Solid Phase Peptide Synthesis, Pierce Chemical Comp., Rockford, Ill. (1984); Atherton et al., Solid Phase Peptide Synthesis: A Practical Approach, IRL: Oxford, (1989)) or on a silyl-linked resin by alcohol attachment (see Randolph et al., J Am. Chem. Soc. 117:5712-14 (1995)).

Other suitable C5a receptor antagonists are reported in U.S. Pat. No. 5,807,824 (van Oostrum et al.) and U.S. Pat. No. 5,837,499 (van Oostrum et al.), both of which are incorporated herein by reference. These patents disclose polypeptide analogues of C5a that exhibit substantially no anaphylatoxin or agonist activity. These C5a receptor antagonists differ from human C5a via two modifications made by mutagenizing the portion of a synthetic C5a gene encoding the C-terminal region, i.e., amino acids 64-74, of human C5a. The C5a receptor antagonists are truncated at least to Leu (72); i.e., by removing the Gly (73) and Arg (74) residues, and at least one cysteine is substituted in the C-terminal region, provided that the C-terminal amino acid of the polypeptide (i.e., the C-terminus) is cysteine, and that the thiol (SH) group of the C-terminal cysteine is in reduced form (i.e., has a free thiol group), or is in a form capable of spontaneously converting or being readily converted into a free thiol group.

Other C-5a receptor antagonists that may be useful in the present invention are organic molecules disclosed in U.S. Pat. Nos. 6,723,743, 6,777,422, 6,858,637 and 6,884,815.

Typically, doses of C5a receptor antagonists, such as 3D53, range from about 0.1 mg/kg to about 20 mg/kg, and preferably from about 1 mg/kg to about 10 mg/kg. See Strachan A J, Br. J. Pharmacol., 134::1778 (2001); Strachan A J, J. Immunol., 164:6560 (2000); Woodruff T M, Arthritis Rheum., 4:2476 (2002); and Woodruff T M, J. Immunol., 171:5514 (2003).

In the present invention, at least one C5a inhibitor is administered to a pregnant mammal. Different C5a inhibitors may be administered in any one administration or during the course of treatment. The different C5a inhibitors may be from the same class of inhibitors or from different classes. For example, one or more antibodies that specifically bind the C5a moiety of native C5 and/or free C5a, and one or more C5a receptor antagonists may be co-administered (as used herein).

The C5a inhibitor, any combination thereof, or other active agents of the present invention may be administered at anytime during pregnancy and particularly, when the need for such treatment arises. For example, Pexelizumab is relatively short acting with an elimination half life of approximately 7.0 to 14.5 hours, the latter only after an initial dose of 2 mg/kg. Follow-up doses of 0.05 mg/kg may be administered after the initial dose in hourly increments. However, Eculizumab is a relatively long acting molecule with a longer elimination half life. Accordingly, Pexelizumab is preferred for acute treatment of severe preeclampsia remote from term, and Eculizumab, is preferred as a protective/preventive agent to avert the inflammatory symptoms of preeclampsia in patients identified early in pregnancy at risk for the disease.

While not intending to be bound by theory, it is believed that the C5 inhibitors of the present invention do not interfere with the activation of complement C3, which is upstream in the classical pathway of the complement cascade. Blocking the complement cascade at C5, C5a or the C5a receptor allows for treatment or prevention of preeclampsia while preserving the complement derived immunoprotective effects of complement C3.

In some embodiments of the present invention, the C5a inhibitor is co-administered with at least one other active agent. As used herein, the term “co-administered” refers to administration of C5a inhibitor and any other active agent(s) in the same course of treatment. Thus, the administration of the different agents need not take place via the same dosage formulation or even at the same time, generally they would be administered in the same 24-hour period. Generally, the C5a inhibitor and any active agents are both administered within the same twenty four-hour period. The C5a inhibitor and any active agents may be administered simultaneously or at different times within the same twenty four-hour period. If administered separately within the twenty four-hour period, they are preferably administered within about six to about 12 hours of each other.

The other active agents include apoptosis inhibitors, coagulation inhibitors, immune complex inhibitors, immune complex production inhibitors, anti-inflammatory agents, granulocyte activation inhibitors, antioxidants, serotonin/histamine inhibitors, platelet activation inhibitors, and anti-hypertensive agents. These active agents may be co-administered with the C5a inhibitor in the same formulation, or separately. For purposes of the present invention, the combination therapy is referred to as a therapeutic cocktail or combination.

Apoptosis is a highly regulated process of programmed cell death that is essential to the health and homeostasis of a given tissue by eliminating superfluous, damaged, mutated, or aged cells. The process is orchestrated by the activation of cysteine aspartate-specific proteases, or caspases, via two distinct signaling pathways: a mitochondrial-cytochrome C (receptor independent) pathway, and a ligand-death receptor dependent pathway. In the ligand-death receptor pathway, ligation of death receptors induces the formation of a death inducing signaling complex (DISC) resulting in apoptosis. Innate regulation of apoptosis is via separate pro- and anti-apoptotic members of the Bcl-2 protein family in the mitochondrial pathway. Also, natural inhibitors of caspases, coined the “inhibitors of apoptosis proteins (IAPs),” are found in cells and exhibit anti-apoptotic activity to a broad range of stimuli in both pathways.

In pregnancies complicated by preeclampsia, there is an increased level of placental apoptosis. See Leung et al., Increased placental apoptosis in pregnancies complicated by preeclampsia, Am. J. Obstet. Gynecol. 184:1249-50 (2001); Allaire et al., Placental apoptosis in preeclampsia, Obstet. Gynecol. 96:271-6 (2000); Ishihara et al., Increased apoptosis in the syncytiotrophoblast in human term placentas by either preeclampsia or intrauterine growth retardation, Am. J. Obstet. Gynecol. 186:158-66 (2002); and Crocker et al., Differences in apoptotic susceptibility of cytotrophoblasts and syncytiotrophoblasts in normal pregnancy to those complicated with preeclampsia and intrauterine growth restriction, Am. J. Pathol. 162:637-43 (2003). By decreasing placental apoptosis in cases of advanced clinical preeclampsia, immune complex production is decreased, resulting in a lower proinflammatory burden for the mother.

Clinically, heparin and aspirin are widely used with success as treatment for pregnant patients with antiphospholipid antibodies (APLAS) As reported in Bose et al., Heparin and aspirin attenuate placental apoptosis in vitro: implications for early pregnancy failure, Am. J. Obstet. Gynecol. 192:23-30 (2005), which is incorporated herein by reference, in vitro studies demonstrate heparin and aspirin regulate trophoblast apoptosis. Thus, administration of such agents may is reduce maternal trophoblast apoptotic burden.

Other apoptosis inhibitors that may be co-administered with the C5a inhibitors include caspase inhibitors, which are agents that inhibit or interrupt signaling along either of the two apoptosis pathways, the mitochondrial-cytochrome C (receptor independent) pathway and the ligand-death receptor dependent pathway. Such caspase inhibitors include IDN-5370 (Idun Pharmaceuticals, Inc.), VX-799 (Vertex Pharmaceutials, Inc.), M-920 (Merck Frosst), M-791 (Merck Frosst) and Caspase 8 inhibitors. IDN-5370 is a peptidomimetic caspase inhibitor from the structural class of oxoazepinoindoline caspase inhibitors; VX-799 is a small molecule caspase inhibitor; M-920 is a broad-spectrum caspase inhibitor; and M-791 is a highly selective caspase-3 inhibitor.

Typically, apoptosis inhibitors are administered in dosage amounts ranging from between about 0.1 mg per kg and about 100 mg per kg, and preferably about 5 mg per kg to about 50 mg per kg.

Other active agents for use in the present invention are immune complex production reducing agents (e.g., B cell attenuators). Normally, the natural means of achieving a decrease in immune complex production is either delivery of the placenta, or having more children with the same paternity (i.e., induction of immune tolerance in multigravidas). Medical therapy to decrease the immune complex load might include agents to decrease maternal antibody production. As reported in Looney et al., B cells as therapeutic targets for rheumatic diseases, Curr. Opin. Rheumatol., 16:180-5 (2004), which is incorporated herein by reference, pharmacologic interruption of B cell antibody production can be accomplished via CD20 monoclonal antibodies. For example, Rituximab® (Roche Pharmaceuticals, Nutley, N.J.) is a monoclonal antibody in clinical use for treatment of autoimmune disease and several types of non-Hodgkin's lymphoma. The antibody binds to the B cell surface protein CD20 triggering the body's immune system to attack and destroy the cell. Since normal B cells are quickly replaced, toxicity is low. In the case of preeclampsia, a low dose (e.g., about 200 mg/m²/week to about 500 mg/m²/week (preferably, about 375 mg/m²/week)) Rituxumab would be effective to decrease the B cell antibody load enough to achieve a decrease in immune complex production.

Other active agents that may be co-administered with a C5a inhibitor are anti-inflammatory agents. Suitable anti-inflammatory agents include corticosteroids, also known as “steroids.” Generally, steroids act at the genetic level and result in down-regulation of immune pathways and various proinflammatory mediators, such as cytokines. See Van der Velden, V H, Glucocorticoids: mechanisms of action and anti-inflammatory potential in asthma, Mediators Inflamm., 7:229-37 (1998). Steroids useful in the present invention include cortisone, hydrocortisone, prednisolone, prednisone, methylprednisolone, budesonide, betamethasone, dexamethasone and beclomethasone.

Typically, steroids are administered in dosage amounts between about 0.1 mg/kg and about 50 mg/kg per day, and preferably about 0.1 mg/kg to about 5 mg/kg per day.

Other anti-inflammatory compounds, such as monoclonal anti-cytokine antibodies, tumor necrosis factor-α (TNF-α) antibodies, soluble tumor necrosis factor (TNF) receptors (fusion proteins that cause TNF inhibition), interleukin-1 receptor antagonists interleukin-1 beta-converting enzyme (ICE) inhibitors and p38 mitogen-activated protein kinase (MAP kinases) inhibitors may also be useful. There have been reports that in patients with preeclampsia, there is a deficiency of placental and serum inhibitory cytokine levels, such as interleukin-10. See Hennessy et al., A deficiency of placental IL-10 in preeclampsia, J. Immunol. 163:3491-5 (1999); and Orange et al., Preeclampsia is associated with a reduced interleukin-10 production from peripheral blood mononuclear cells, Hypertens Pregnancy 22:1-8 (2003). Thus, inhibitory cytokines may also be suitable as anti-inflammatory agents for use in the present invention.

Typically, TNF-α antibodies are administered in amounts ranging from about 20 mg to about 60 mg once every other week, and preferably about 40 mg once every other week; TNF receptors are administered in amounts ranging about 10 mg to about 50 mg twice weekly, and preferably about 25 mg twice weekly; ICE inhibitors are administered in amounts ranging about 1 mg/kg to about 2.5 mg/kg; and p38 MAP kinases are administered in amounts ranging about 10 mg/kg to about 50 mg/kg.

Granulocyte activation inhibitors are another class of active agents that may be co-administered with the C5a inhibitors of the present invention. Aside from the classical pathway and its complement activation scheme, a second pathway of immune complex injury is direct activation of granulocytes via their surface receptors FcgRI and FcgRIII. These granulocyte receptors are proinflammatory while FcgRIIB exhibits inhibition of inflammatory processes. Traditionally, immune complexes were thought to cause inflammatory effects only via complement activation. More recently, however, it has become clear that direct activation of effector cells by immune complexes is intricately involved in their inflammatory sequelae, with granulocyte Fcg receptors playing the pivotal role in this pathway. Fcg receptors are surface glycoproteins, or members of the immunoglobulin gene superfamily of proteins, that can bind the Fc portion of immunoglobulin molecules. Fcg receptor expression is under the redundant control of numerous cytokines and genetic factors. See Ravetch, J V, A full complement of receptors in immune complex diseases., J. Clin. Invest. 110:1759-61 (2002). Both activating and inhibitory signals are transduced through the Fcg receptors following ligation. These diametrically opposing functions result from structural differences among the different receptor isoforms. Two distinct domains within the cytoplasmic signaling domains of the receptor called immunoreceptor tyrosine based activation motifs (ITAMs) or immunoreceptor tyrosine based inhibitory motifs (ITIMS) account for the different responses. The recruitment of different cytoplasmic enzymes to these structures dictates the outcome of the Fcg receptor-mediated cellular responses.

The balance between activation proinflammatory receptors (FcgRI and FcgRIII) and inhibition receptors (FcgRIIB) is critical to the net immune response. Upregulation of the activation FcgRIII, induced by IFN-g or C5a, results in a lowered threshold for immune complex stimulation and consequently an enhanced inflammatory response. Conversely, upregulation of the inhibitory FcgRIIB molecule raises the threshold for immune complex stimulation and suppresses inflammatory response to IgG antibodies. Recent data implicate the cell surface density of FcgRIIB as potentially the immune response “gatekeeper” in this balance. See McGaha et al., Restoration of tolerance in lupus by targeted inhibitory receptor expression, Science 307:590-3 (2005). Pharmacologic administration of intravenous immune globulin (IVIG) has been shown to induce inhibitory FcgRIIB expression, thus raising the threshold for immune complexes to trigger FcgRIII activation. See Samuelsson et al., Anti-inflammatory activity of IVIG mediated through the inhibitory Fc receptor, Science 291:484-6 (2001).

In addition, the complement system itself can influence Fcg receptor activity. Both complement and IgG Fc receptors interact in vivo with C5a acting as an early regulator of the induction of activating FcgRIII and suppression of the inhibitory FcgRII. See Shushakova et al., C5a anaphylatoxin is a major regulator of activating versus inhibitory FcgRs in immune complex-induced lung disease, J. Clin. Invest. 110:1823-30 (2002). Thus, in addition to regulation of activation FcgRIII by C5a blockade, pharmacologic upregulation of inhibition FcgRIIB by IVIG may serve to alter the threshold of immune complex mediated inflammation and injury. Other strategies to influence Fcg receptor activity include statin therapy(Hillyard et al., Fluvastatin inhibits raft dependent Fcg receptor signaling in human monocytes, Atherosclerosis 172:219-28 (2004)) and Fcg receptor specific antibodies (MacroGenics Inc, Rockville, Md.).

Suitable granulocyte activation inhibiting agents for use in the present invention include intravenous immune globulin (IVIG) and statins, such as fluvastatin and pravastatin and atorvastatin.

Other suitable active agents are anti-coagulants. Normally, at rest, the endothelial surface is essentially non-thrombogenic. This state is largely maintained by the tissue factor pathway inhibitor (TFPI), which blocks the initiation of blood coagulation by tissue factor. Endothelial cells are the main source of TFPI. There exists an intricate interrelationship between the coagulation system and host inflammatory response. For example, inflammatory cytokines can activate coagulation and inhibit fibrinolysis, whereas thrombin is able to stimulate multiple inflammatory pathways. As shown in Weiner, C P, Preeclampsia-eclampsia syndrome and coagulation, Clin. Perinatol. 18:713-26 (1991), the coagulation cascade is activated in patients with preeclampsia. With severe maternal preeclamptic disease, a marked consumptive coagulopathy and thrombopathy can manifest as disseminated intravascular coagulation.

Accordingly, one or more anticoagulants may be administered to reduce or prevent any disseminated intravascular coagulation present during preeclampsia. Suitable anticoagulants include heparin, aspirin, recombinant TFPI (tifacogin, Chiron Corp, Emeryville, Calif.), recombinant activated protein C (drotrecogin alfa/Xigris, Eli Lilly, Indianapolis, Ind.), ximelagatran (Exanta) (AstraZeneca, Waltham, Mass.), dalteparin/Fragmin (Pfizer, New York, N.Y.) and enoxaparin/warfarin (Sanofi-Aventis Pharmaceuticals, Bridgewater, N.J.).

Typical administration amounts for enoxaparin range from about 0.5 mg/kg to about 2 mg/kg. Specifically, the administration amounts generally are about 1 mg/kg once or twice daily for enoxaparin (Lovenox); about 2500 to about 5000 International Units (IU) daily for dalteparin (Fragmin). Unfractionated heparin is generally dosed between about 2500 to about 7500 units once or twice daily; about 81 mg daily for aspirin; about 0.1 mg/kg/hr to about 0.5 mg/kg/hour for 96 hours, and preferably about 0.25 mg/kg/hr for 96 hours, for tifacogin; about 10 ug/kg/hr to about 50 ug/kg/hr for 96 hours for drotrecogin; and about 24 mg orally twice daily for ximelagatran.

Preeclampsia is also associated with an increased production of reactive oxygen species as a result of the inherent ongoing inflammatory process. In preeclampsia, neutrophils which have accumulated in inflammatory sites are activated by C3b-opsonized immune complexes. They release toxic oxygen radicals which cause further damage, or “oxidative stress.” Adjuvant administration of antioxidant therapy may ameliorate these clinical symptoms. In one randomized trial, supplementation with daily Vitamin C (1000 mg) and Vitamin E (400 IU) was associated with a 54% reduction in the rate of preeclampsia in women identified as being at high risk for preeclampsia. See Chappell et al., Effect of antioxidants on the occurrence of pre-eclampsia in women at increased risk: a randomised trial, Lancet. 354:810-6 (1999). This antioxidant therapy was also associated with improvement in the biochemical indices of preeclamptic oxidative stress. See Chappell et al., Vitamin C and E supplementation in women at risk of preeclampsia is associated with changes in indices of oxidative stress and placental function, Am. J. Obstet. Gynecol. 187:777-84 (2002). In addition to Vitamin C and Vitamin E, other anti-oxidants include Vitamin A, beta-carotene and mineral selenium.

Typically, administration amounts for anti-oxidants are: about 75 mg to about 2000 mg daily for vitamin C; about 22 IU to about 1500 IU daily for vitamin E; about 55 mg to about 400 mg daily for selenium; and about 1000 IU to about 10,000 IU daily, and preferably 5000 IU daily, for vitamin A.

In preeclampsia, immune complexes and anaphylatoxins cause the release of histamine from basophils and mast cells and serotonin from platelets (via activated leukocytes resulting in endothelial cell retraction that leads to increased vascular permeability and clinical edema.

Serotonin (5-hydroxytryptamine, 5HT) is formed by the hydroxylation and decarboxylation of tryptophan. The greatest concentration of 5HT (90%) is found in the enterochromaffin cells of the gastrointestinal tract. Most of the remainder of the body's 5HT is found in platelets and the CNS. The effects of 5HT are felt most prominently in the cardiovascular system, with additional effects in the respiratory system and the intestines. Vasoconstriction is a classic response to the administration of 5HT. The function of serotonin is exerted upon its interaction with specific receptors. Several serotonin receptors have been cloned and are identified as 5HT₁, 5HT₂, 5HT₃, 5HT₄, 5HT₅, 5HT₆, and 5HT₇, Within the 5HT₁ group there are subtypes 5HT_{1A}, 5HT_{1B}, 5HT_{1D}, 5HT_{1E}, and 5HT_{1F}. There are three 5HT₂ subtypes, 5HT_{2A}, 5HT_{2B}, and 5HT_{2C} as well as two 5HT₅ subtypes, 5HT₅ₐ and 5HT_{5B}. Most of these receptors are coupled to G-proteins that affect the activities of either adenylate cyclase or phospholipase Cg. The 5HT_{2A} receptors mediate platelet aggregation and smooth muscle contraction.

Serotonin/histamine blocking agents may be administered with the C5a inhibitor. The serotonin/histamine blocking agents may be in the form of a combination of individual serotonin and histamine agents or one agent that blocks both histamine and serotonin.

Examples of histamine blockers include: diphenhydramine (Benadryl, loratadine (Claritin), fexofenadine (Allegra), cetirizine (Zyrtec), terfenadine (Seldane). Examples of serotonin blockers include: sarpogrelate, LY53857, sergolexole, imipramine, nefazodone, and mirtazipine. Dosage ranges for these histamine and serotonin blockers are known in the art and are per standardized published manufacturer recommendations.

An example of a combined histamine and serotonin blocker is cyproheptadine hydrochloride (PeriActin). The typical dosage range for combined histamine and serotonin blockers is from about 2 mg to 8 about 8 mg four times daily.

Preeclampsia may also involve binding of platelets to exposed subendothelial collagen, aggregation to form microthrombi, and activation of the coagulation cascade, releasing more bioactive mediators. Thus, antiplatelet agents may be co-administered. In particular, a recent Cochrane review found that the risk of preeclampsia associated with the use of antiplatelet drugs decreased by 15% and the risk of neonatal mortality decreased 14%, and concluded that there are benefits to the administration of antiplatelet agents, principally low dose aspirin, in the prevention of preeclampsia. See Knight et al., Antiplatelet agents for preventing and treating pre-eclampsia, The Cochrane Database of Systematic Reviews, Issue 2, Art. No.: CD000492, DOI:10.1002/14651858, CD000492 (2002). In addition to aspirin, other suitable anti-platelet agents include dipyridamole (Persantine, Boehringer Ingleheim), administered in a dosage of about 75 mg to about 100 mg four times daily; tirofiban (Aggrastat, Merck, which typically administered in an initial dose ranging from about 0.1 ug/kg/min to about 1.0 ug/kg/min, and preferably 0.4 ug/kg/min for a 30 min bolus, that is then followed by maintenance dosages of approximately 0.1 ug/kg/min; and clopidogrel (Plavix) (Bristol-Myers Squibb/Sandofi), which is typically administered in a loading dose of about 300 mg and then continued with a dosage of about 75 mg once daily.

Anti-hypertensive agents may also be used in the present invention to reduce or prevent hypertension associated from preeclampsia. One suitable anti-hypertensive agent, Labetalol (sold under the trade names Trandate® and Normodyne®, blocks receptors of the adrenergic nervous system, the system of nerves in which epinephrine (adrenalin) is active. Nerves from the adrenergic system within the arteries release an adrenergic chemical (norepinephrine) that attaches to the receptors on the muscles of the arteries and causes the muscles to contract, which narrows the arteries and increases blood pressure. Labetalol is believed to attach to and block the receptors, allowing the arterial muscles to relax and the arteries to expand, resulting in a fall in blood pressure.

Another suitable anti-hypertensive agent, nifedipine (sold under the brand names Adalat® and Procardia®), belongs to a class of medications called calcium channel blockers. These medications are believed to block the transport of calcium into the smooth muscle cells lining the coronary arteries and other arteries of the body. Since calcium is important in muscle contraction, blocking calcium transport relaxes artery muscles and dilates coronary arteries and other arteries of the body.

Yet another suitable anti-hypertensive agent for use in the present invention is Ketanserin, a serotonin receptor antagonist. See Steyn et al., Randomised controlled trial of Ketanserin and aspirin in prevention of pre-eclampsia, Lancet. 350:1267-71 (1997); and Bolte et al., Ketanserin versus dihydralazine in the management of severe early-onset preeclampsia: maternal outcome, Am. J. Obstet. Gynecol. 80:371-7 (1999). Ketanserin has a high affinity for the serotonin 5-HT2A receptor but also binds less potently to the 5-HT2C, 5-HT2B, 5-HT1D, alpha-adrenergic, and dopamine receptors. Serotonin receptor antagonists, such as Ketanserin, bind to but do not activate serotonin receptors, thereby blocking the actions of serotonin or serotonin agonists. As a result, Ketanserin inhibits serotonin-induced platelet aggregation and lowers blood pressure.

Methyldopa (Aldomet) is another suitable anti-hypertensive agent for use in the present invention. Methyldopa is an aromatic-amino-acid decarboxylase inhibitor, and has been shown to cause a net reduction in the tissue concentration of serotonin, dopamine, norepinephrine, and epinephrine.

Dosage ranges for these antihypertensive agents are known in the art and are per standardized published manufacturer recommendations.

Broadly, the method of the present invention can be used at anytime during pregnancy. When a mammal is at term or past term, delivery remains a first option, if practical. Thus, use of the present invention is preferred at other times during pregnancy (e.g., early in pregnancy or remote from term such as in cases of marked prematurity). As used herein, “at term” refers to the end of the gestation period, which is typically beyond thirty seven completed gestational weeks. Any period of time after forty two completed gestational weeks is considered “past term” or “post-term.” An example of a therapeutic cocktail for cases of marked prematurity is soluble CR1 (TP-10, Avant Immunotherapeutics) or a C5 binding antibody, such as Pexelizumab, for complement regulation, intravenous immune globulin for upregulation of the inhibitory FcgRIIB, corticosteroid(s) for immune modulation, an anti-hypertensive agent (e.g., Ketanserin, Labetalol, nifedipine, or Aldomet), low molecular weight (e.g., about 4000 to about 6500 daltons) heparin/low dose aspirin for inhibition of the clotting cascade as well as inhibition of placental apoptosis, an antioxidant (e.g., Vitamin C & E) therapy, and a serotonin/histamine blocking agent.

The method of the present invention can be used even when there is no confirmed diagnosis of preeclampsia, such as in the cases when a pregnant mammal is identified as being at risk for preeclampsia. Thus, the method of the present invention may be practiced prophylactically as well. A pregnant mammal without preeclampsia, e.g., suspected at being at risk for preeclampsia, may be screened for preeclampsia using standard techniques. A number of tests are available for such screenings. See Conde-Agudeloet al., World Health Organization systematic review of screening tests for preeclampsia, Obstet. Gynecol. 104:1367-91 (2004). Markers used in screenings for determining risk for preeclampsia include: log[sFlt-1/P1GF] ratio (Buhimschiet al., Urinary angiogenic factors cluster hypertensive disorders and identify women with severe preeclampsia, Am. J. Obstet. Gynecol. 192:734-41 (2005)), placental growth factor (Levine et al., Urinary placental growth factor and risk of preeclampsia, JAMA 293:77-85 (2005)), cell free fetal DNA concentration (Levine et al., Two-stage elevation of cell-free fetal DNA in maternal sera before onset of preeclampsia, Am. J. Obstet. Gynecol. 190:707-13 (2004)), uterine artery Doppler velocimetry (Harrington et al., Transvaginal uterine and umbilical artery Doppler examination of 12-16 weeks and the subsequent development of pre-eclampsia and intrauterine growth retardation, Ultrasound Obstet. Gynecol. 9:94-100 (1997)), PAPP-A levels (Bersinger et al., Women with preeclampsia have increased serum levels of pregnancy-associated plasma protein A (PAPP-A), inhibin A, activin A and soluble E-selectin, Hypertens. Pregnancy 22:45-55 (2003)), erythrocyte CR1 levels (Feinberg et al., Decreased erythrocyte C3b receptor (CR1) expression in preeclamptic gestations, Soc. Gyn. Invest. Abstract P263 (1993), and breath markers of oxidative stress (Moretti et al., Increased breath markers of oxidative stress in normal pregnancy and in preeclampsia, Am. J. Obstet. Gynecol. 190:1184-90 (2004). If the screening demonstrates a risk for preeclampsia, one or more C5a inhibitors, alone or as part of a therapeutic cocktail, may be administered prophylactically. One example of a prophylactic regimen includes: oral administration of 3D53 or weekly Eculizumab injection, heparin/low dose aspirin or Exanta, antioxidants (e.g., Vitamins C and E), a serotonin and histamine blocking agent, and optionally, steroids (e.g., prednisone).

Administration of the C5a inhibitors and other active agents may be performed by an intravascularly, e.g., via intravenous infusion by injection. Formulations suitable for intravascular delivery are disclosed in Remington's Pharmaceutical Sciences, Mack Publishing Company, Philadelphia, Pa., 17th ed. (1985). Such formulations must be sterile and non-pyrogenic, and generally will include a pharmaceutically effective carrier, such as saline, buffered (e.g., phosphate buffered) saline, Hank's solution, Ringer's solution, dextrose/saline, glucose solutions, and the like. The formulations may contain pharmaceutically acceptable auxiliary substances as required, such as, tonicity adjusting agents, wetting agents, bactericidal agents, preservatives, stabilizers, and the like.

Other routes of administration may be used if desired or practical under the circumstances. For some C5a inhibitors, such as 3D53 and NGD-1000, oral administration is an option, and may in some cases be preferred because of its greater convenience and acceptability. Formulations or compositions intended for oral use may be prepared according to methods known to the art. Such compositions may contain one or more agents selected from the group consisting of sweetening agents, flavoring agents, coloring agents and preserving agents in order to provide pharmaceutically elegant and palatable preparations, and include a pharmaceutically acceptable carrier. The oral administrations may be in the form of a pill/tablet, capsule (e.g., gelcaps), elixir, syrup, suspension lozenge or troche. Syrups and elixirs may be formulated with sweetening agents, for example glycerol, propylene glycol, sorbitol or sucrose. Such formulations may also contain a demulcent, a preservative, and flavoring and coloring agents.

In tablet form, the formulations contain the one active ingredient in admixture with non-toxic, pharmaceutically acceptable excipients that are suitable for the manufacture of tablets. For example, these excipients may be inert diluents, such as calcium carbonate, sodium carbonate, lactose, calcium phosphate or sodium phosphate; granulating and disintegrating agents (e.g., corn starch or alginic acid); binding agents (e.g., starch, gelatin or acacia); and lubricating agents (e.g., magnesium stearate, stearic acid or talc). The tablets may be uncoated or they may be coated by known techniques to delay disintegration and absorption in the gastrointestinal tract and thereby, provide a sustained action over a longer period. For example, a time delay material such as glyceryl monosterate or glyceryl distearate may be employed.

Formulations for oral use may also be presented as hard gelatin capsules, in which the active ingredient is mixed with an inert solid diluent (e.g., calcium carbonate, calcium phosphate or kaolin) or as soft gelatin capsules, in which the active ingredient is mixed with water or an oil medium (e.g., peanut oil, liquid paraffin or olive oil).

Aqueous suspensions contain the active materials in admixture with excipients suitable for the manufacture of aqueous suspensions. Such excipients may be suspending agents (e.g., sodium carboxymethylcellulose, methylcellulose, hydropropylmethylcellulose, sodium alginate, polyvinylpyrrolidone, gum tragacanth and gum acacia) or dispersing or wetting agents, such as a naturally-occurring phosphatide (e.g., lecithin), condensation products of an alkylene oxide with fatty acids (e.g., polyoxyethylene stearate), condensation products of ethylene oxide with long chain aliphatic alcohols (e.g., heptadecaethyleneoxycetanol), condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives (e.g., ethyl or n-propyl p-hydroxybenzoate), one or more coloring agents, one or more flavoring agents, and one or more sweetening agents, such as sucrose or saccharin.

Oily suspensions may be formulated by suspending the active ingredients in a vegetable oil, such as arachis oil, olive oil, sesame oil or coconut oil, or in a mineral oil, such as liquid paraffin. The oily suspensions may contain a thickening agent, such as beeswax, hard paraffin or cetyl alcohol. Sweetening agents, such as those set forth above, and flavoring agents may be added to provide palatable oral preparations. These compositions may be preserved by the addition of an antioxidant such as ascorbic acid.

Suitable dispersible powders and granules for the aqueous suspension are prepared by the addition of water, and provide the active ingredient in admixture with a dispersing or wetting agent, suspending agent and one or more preservatives. Suitable dispersing or wetting agents and suspending agents are exemplified by those already mentioned above. Additional excipients, such as sweetening, flavoring and coloring agents, may also be present.

Oral formulations of the invention may also be in the form of oil-in-water emulsions. The oily phase may be a vegetable oil (e.g., olive oil or arachis oil), a mineral oil (e.g., liquid paraffin) or mixtures therof. Suitable emulsifying agents may be naturally occurring gums (e.g., gum acacia or gum tragacanth), naturally-occurring phosphatides (e.g., soy bean, lecithin, and esters or partial esters derived from fatty acids and hexitol), anhydrides (e.g., sorbitan monoleate), and condensation products of partial esters with ethylene oxide (e.g., polyoxyethylene sorbitan monoleate). The emulsions may also contain sweetening and flavoring agents.

Dosage levels of active ingredients in the formulations of this invention may be varied (e.g., within or outside of any specific ranges disclosed herein) so as to obtain an amount of the active compound(s) that is effective to achieve the desired therapeutic response for a particular patient, compositions, and mode of administration. The selected dosage level will depend upon the activity of the particular compound, the route of administration, the severity of the condition, and the condition and prior medical history of the patient. However, it is within the skill of the art to initiate dosing of the C5a inhibitors at levels lower than required for to achieve the desired therapeutic effect and by increase the dosage until the desired effect is achieved.

All publications cited in the specification are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method for treating a pregnant mammal afflicted with or at risk of preeclampsia, comprising:
administering to a pregnant mammal in need thereof, an effective amount of at least one C5a inhibitor.

2. The method of claim 1, wherein said C5a inhibitor is an antibody that specifically binds the C5a moiety of C5, but does not substantially bind free C5a.

3. The method of claim 2, wherein said antibody is a 5G1.1.

4. The method of claim 3, wherein said 5G1.1 is humanized.

5. The method of claim 4, wherein said 5G1.1 is a single chain antibody.

6. The method of claim 1, wherein said C5a inhibitor is an antibody that specifically binds the C5a moiety of C5 and free C5a.

7. The method of claim 6, wherein said antibody is MAb 137-26.

8. The method of claim 1, wherein said C5a inhibitor is a C5a receptor antagonist.

9. The method of claim 8, wherein said C5a receptor is macrocycle Ac-Phe(Orn-Pro-D-Cha-Trp-Arg).

10. The method of claim 9, wherein said macrocycle Ac-Phe(Orn-Pro-D-Cha-Trp-Arg) is administered orally.

11. The method of claim 1, wherein said C5a inhibitor is co-administered with at least one other active agent selected from the group consisting of apoptosis inhibitors, coagulation inhibitors, immune complex inhibitors, immune complex production inhibitors, anti-inflammatory agents, granulocyte activation inhibitors, antioxidants, serotonin/histamine inhibitors, platelet activation inhibitors and anti-hypertensive agents.

12. The method of claim 11, wherein said other active agent is Vitamin C or Vitamin E.

13. The method of claim 11, wherein said other active agent is a serotonin/histamine inhibitor comprising cyproheptadine hydrochloride.

14. The method of claim 11, wherein said other active agent is an anti-hypertensive agent comprising Labetalol, Ketanserin, nifedipine or Aldomet.

15. The method of claim 1, wherein said C5a inhibitor is co-administered with at least one intravenous immune globulin, steroid, anti-hypertensive agent, heparin or aspirin, antioxidant and a serotonin/histamine inhibitor.

16. The method of claim 1, wherein said C5a inhibitor is co-administered with at least one of heparin or aspirin or macrocycle Ac-Phe(Orn-Pro-D-Cha-Trp-Arg) or 5G1.1, and at least one of Vitamin C, Vitamin E and a serotonin/histamine inhibitor.

17. The method of claim 1, wherein said C5a inhibitor is co-administered with at least one of heparin or aspirin or macrocycle Ac-Phe(Orn-Pro-D-Cha-Trp-Arg) or 5G1.1, and at least one of Vitamin C, Vitamin E, serotonin/histamine inhibitor and a steroid.

18. A therapeutic cocktail for treatment of a pregnant mammal afflicted with or at risk of preeclampsia, comprising an effective amount of at least one C5a inhibitor and at least one active agent selected from the group consisting of apoptosis inhibitors, coagulation inhibitors, immune complex inhibitors, immune complex production inhibitors, anti-inflammatory agents, granulocyte activation inhibitors, antioxidants, serotonin/histamine inhibitors, platelet activation inhibitors and anti-hypertensive agents.

19. The therapeutic cocktail of claim 18, which in addition to said C5a inhibitor, further comprises an intravenous immune globulin, steroid, anti-hypertensive agent, heparin or aspirin, antioxidant, and serotonin/histamine inhibitor.

20. The therapeutic cocktail of claim 18, which in addition to said C5a inhibitor, further comprises at least of one of heparin or aspirin or macrocycle Ac-Phe(Orn-Pro-D-Cha-Trp-Arg) or 5G1.1, and at least one of Vitamin C, Vitamin E, and a serotonin/histamine inhibitor.

21. The therapeutic cocktail of claim 20, further comprising a steroid.
